(19) United States
(12) Patent Application Publication
(10) Pub. No.: US 2026/0207670 A1
(43) Pub. Date: Jul. 23, 2026

(54) STABILIZED BLOOD DERIVED COMPOSITION USING ANTIFIBRINOLYTIC AGENTS

(71) Applicant: Antoine Turzi, Lauenen b. Gstaad (CH)

(72) Inventor: Antoine Turzi, Lauenen b. Gstaad (CH)

(21) Appl. No.: 19/134,741

(22) PCT Filed: Dec. 21, 2023
(86) PCT No.: PCT/EP2023/087473

§ 371 Date: Jun. 1, 2025

(30) Foreign Application Priority Data
Dec. 22, 2022 (GB) 2219505.1

(51) Int. Cl. A61K 35/19; A61J 1/05; A61K 35/16; A61K 47/18; A61K 47/36

(52) U.S. Cl. CPC A61K 35/19; A61J 1/05; A61K 35/16; A61K 47/183; A61K 47/36

(57) ABSTRACT

The present invention relates to a centrifugation container comprising an antifibrinolytic substance. Also provided is a medical composition comprising platelet rich plasma, platelet rich fibrin, bone marrow concentrate or a combination thereof and; an antifibrinolytic substance.

The present invention relates to centrifugation containers comprising an antifibrinolytic substance for use in the preparation of standardized and stabilized blood and bone marrow compositions/gels. Medical compositions obtained using the centrifugation containers are also provided and include platelet rich plasma, platelet rich fibrin, bone marrow concentrate or a combination thereof; and an antifibrinolytic substance, as well as processes for using the centrifugation containers. The compositions are of use in therapy, in particular for the treatment or prevention of a joint disorder or condition by providing a longer clinical efficacy, as evidenced by the prolonged release of platelet growth factors.

### BACKGROUND OF INVENTION

The importance of biological autologous materials in the healing process has been well documented. Most importantly, two biological autologous materials have been shown to be directly implicated in the formation of the structure of blood clots, which provide a haemostatic barrier whose role is to ensure haemostasis and seal the wound: (1) fibrin, which derives from the separation of plasma fibrinogen into two strands through the action of thrombin, and (2) the activated membranes of platelets. The wound healing process is generally presented as the succession of a coagulation phase, an inflammatory process and a regeneration process. The coagulation phase (blood clotting or clot formation) is a complex process whereby a damaged blood vessel wall is covered by a fibrin clot to stop haemorrhage and the repair of the damaged vessel is initiated by the release in large quantities of cytokines and growth factors from platelet alpha granules. The formation of blood clots (formed in physiological conditions by fibrin, platelets and red blood cells, among other blood components) is a natural phenomenon that results from tissue trauma and its role in the wound healing process, as well as in the union of bone fractures, is well known.

Blood coagulation is the result of the complex interaction of a number of protein clotting factors through a cascade. In general, damage to the vascular endothelium exposes subendothelial structures, which attract platelets and induce them to aggregate reversibly. The protein thrombin, formed during activation of the coagulation pathway generates insoluble cross-linked fibrils of the protein fibrin and causes the platelets to aggregate irreversibly. The resulting platelet-fibrin clot is an effective barrier against loss of blood from the vascular system and also serves as a scaffold for subsequent repair of the lining of the blood vessel.

Platelet-rich plasma (PRP) can be defined as an autologous concentrate of platelets in a small volume of plasma. It has been developed as an autologous biomaterial and has proven to be useful in the healing and regeneration of tissues (Marx et al., 2004). PRP not only comprises a platelet concentrate but also contains growth factors (such as platelet-derived growth factor (PDGF), vascular endothelial growth factor (VEGF), transforming growth factor (TGF) and epidermal growth factor (EGF)) that are actively secreted by platelets and are known to have a fundamental role in wound healing initiation process. PRP is used in various medical (both therapeutic and cosmetic) applications, in particular in wound and tissue healing.

PRP is typically produced by centrifugation of whole blood to separate the platelets from the red blood cells. Additional centrifugation or separation steps may be used to remove the platelet poor plasma. As the cellular composition of PRP, and hence its therapeutic effect, is strongly dependent on the methodology and device used to prepare the PRP, standardization of production of PRP is essential. Medical devices for obtaining standardized PRP have been developed, including devices of use in an automated procedure in a closed circuit. For example, centrifugation tubes produced by Regen Lab and described in WO2008/023026A2, WO2011/110948A2, WO2013/0613092A2, WO2016/083549A2, WO2019/155391A1 and WO2021/198312A1 (all incorporated by reference herein in their entirety).

The coagulation cascade starts as soon as the blood is outside of the venous flow. An anticoagulant is therefore typically used to prevent coagulation during PRP preparation and to maintain PRP in a liquid state. In PRP intended for therapeutic use on patients, the anticoagulant should be fully reversible and have no ancillary effect on the patient when PRP is reinjected. Citrate-based anticoagulants are most commonly used for PRP preparation. Citrate's anticoagulant effect derives from its binding to calcium ions in the plasma. Free calcium ions are essential cofactors of many activation reactions of the coagulation cascade, hence the binding of calcium to citrate hinders clot formation. This anticoagulation is fully reversible, and when PRP is injected in tissues, the level of calcium is rapidly normalized due to calcium in the extracellular fluid.

Alternatively, PRP can be used in gel form, for example to fill a wound. In this case, coagulation can be triggered exogenously by adding to PRP a coagulation activator such as a source of calcium or activated thrombin or a combination of the two. The resulting fibrin-based clot is an adhesive and conductive matrix which induces recruitment of cells to the injury site. Such cells include growth factors which are beneficial to the wound healing. As such, fibrin-based blood clot formation can be advantageous to the healing process.

As well as being of use in wound healing, there has been a great deal of interest in using PRP to reduce pain and promote healing for a number of disorders and conditions that affect the joints. A healthy joint is composed of two bone ends covered by cartilage (hyaline cartilage). This allows for shock absorption and for the bones to slide over one another with ease, thus ensuring joint mobility. Synovial fluid surrounds the cartilage and acts as a lubricant and source of nutrition for the articular cartilage. It is mainly composed of hyaluronic acid (HA), a glycosaminoglycan which binds water molecules and results in a very viscous solution that gives synovial fluid its shock-absorbing properties. It has been shown that the rheological properties of synovial fluid decrease with age and in patients suffering from osteoarthritis, which may cause symptoms of pain and physical loss of function (Chen et al., 2012).

Viscosupplementation with exogenous hyaluronic acid solution is a known therapy which aims to replace the reduced and fragmented hyaluronic acid in the synovial fluid of osteoarthritis patients. Hyaluronic acid is a safe and well-tolerated product and has no known interactions with other medications. However, some meta-analyses have indicated that the positive effects of hyaluronic acid treatment appear to be modest from a clinical point of view and not long lasting (Lo et al., 2003).

A combined therapy involving PRP and hyaluronic acid has been developed by Regen Lab. In vitro studies have shown that when PRP is incubated with hyaluronic acid, the release of growth factors is increased after 5 days (Iio et al., 2016). When PRP combined with hyaluronic acid coagulates, the structure of the resulting fibrin network is different and has a larger porosity than a fibrin clot without hyaluronic acid. This creates a better environment for cells, allowing easier cell migration and proliferation (Smith et al., 2007). A combination of PRP and HA may result in a more stable scaffold that allows the controlled or enhanced release of growth factors into the surrounding milieu, as well as binding extracellular matrix proteins such as fibronectin, and the migration of cells needed for cartilage repair (Iio et al., 2016).

While some studies have shown that administration of PRP alone can improve joint function, and treatment with PRP can provide better outcomes that conventional treatments such as corticosteroids, beneficial effects are relatively modest. Given the success of using PRP as a treatment for other regenerative indications, further treatment regimens for using PRP to treat or prevent joint disorders and conditions such as osteoarthritis are required.

### SUMMARY OF THE INVENTION

According to a first aspect of the present invention, there is provided a centrifugation container comprising an antifibrinolytic substance.

According to a second aspect of the invention, there is provided a process for preparing a medical composition, comprising the steps of (a) centrifuging whole blood or bone marrow in a centrifugation container as described herein; and then (b) collecting the medical composition.

According to a third aspect of the invention there is provided a medical composition obtained according to a process as described herein.

According to a fourth aspect of the invention there is provided a medical composition comprising: platelet rich plasma, platelet rich fibrin, bone marrow concentrate or a combination thereof; and an antifibrinolytic substance.

According to a fifth aspect of the invention there is provided a medical composition as described herein for use in therapy, in particular for use in treating or preventing a joint disorder or condition.

According to a sixth aspect of the invention there is provided a centrifugation container comprising a corticosteroid, anaesthetic (e.g. bupivacaine), a non-steroidal anti-inflammatory drug (e.g. Ketorolac), an analgesic (e.g. acetaminophen), kartogenin and/or haemoglobin, or a combination thereof.

According to a seventh aspect of the invention there is provided a kit comprising:
a centrifugation container; and
a container comprising an antifibrinolytic substance.

Embodiments and preferences described below with respect to the centrifugation container of the invention apply equally to the processes for using the centrifugation container, and to medical compositions described herein.

### BRIEF DESCRIPTION OF FIGURES

FIG. 1 illustrates various centrifugation containers according to the present invention.

FIGS. 2a and 2b show the diameter of platelet rich plasma gel discs obtained using various preparations (with and without tranexamic acid) at different time points (Example 1).

FIG. 3 shows the release of specific growth factor (PDGF) from different platelet rich plasma gel combinations (Example 2).

FIGS. 4a and 4b show radiological scores obtained in a rabbit model of osteoarthritis (Example 3).

FIG. 5 shows macroscopic scores obtained in a rabbit model of osteoarthritis (Example 3).

FIG. 6 shows microscopic scores obtained in a rabbit model of osteoarthritis (Example 3).

FIG. 7 shows meniscal scores obtained in a rabbit model of osteoarthritis (Example 3).

FIG. 8 shows synovial scores obtained in a rabbit model of osteoarthritis (Example 3).

FIG. 9a shows the expression of Sox9 gene obtained in a rabbit model of osteoarthritis (Example 4).

FIG. 9b shows the expression of Type II Collagen (COLII) obtained in a rabbit model of osteoarthritis (Example 4).

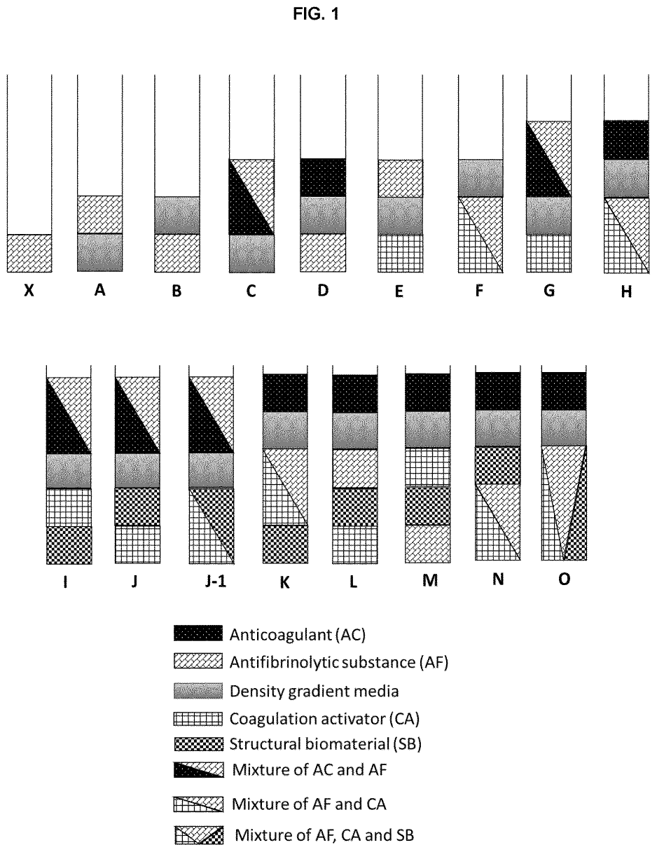

In FIG. 1, it should be noted that the relative proportions of the centrifugation container components are not drawn to scale, and are merely intended to illustrate the relative positions of the components within the container, and do not indicate relative quantities. In addition, the size and shape of the centrifugation container itself is not drawn to scale, and does not include the whole fill volume space.

### DETAILED DESCRIPTION

The present inventors have investigated the potential beneficial effect of using PRP for the treatment of joint disorders and conditions including osteoarthritis, but have obtained only modest results in preclinical trials. Although some beneficial effects have been observed, no significant structural effects in the cartilage and menisci were observed. The inventors hypothesised that it is the instability of fibrin-based matrices in the joint environment that compromises the effectiveness of PRP in the treatment of osteoarthritis. Fibrin-based matrices are broken down during the process of fibrinolysis. The principal enzyme involved in fibrinolysis is plasmin, which acts to dissolve the fibrin clots. Plasmin is derived from plasminogen, which circulates in inactive form until binding to a clot where it is converted to active plasmin. Under certain circumstances, wound healing can be disrupted by premature collapse of the fibrin-based matrix. This reduces the residence time of beneficial growth factors and can prevent or reduce wound healing.

Fibrin-based matrices can also be more or less stable depending on their environment. It is known that fibrin-based matrices are less stable in anterior cruciate ligament (ACL) sites of injury compared with medial collateral ligament (MCL) site of injury. Following an injury, this means that ACL injuries do not heal as well as MCL injuries, or do not heal at all (Woo et al., 2000).

When an antifibrinolytic substance is mixed with liquid PRP, the resulting platelet rich plasma-antifibrinolytic substance can be used to form a gel/clot which provides therapeutic benefits in an osteoarthritis model which are greater than those provided by a platelet rich plasma gel/clot alone (as shown in Examples 1-4). Without being limited by theory, it is believed that the beneficial effect results from the stability of the fibrin clot being prolonged, by counteracting the effects of fibrinolysis. When the fibrin clot is formed in the presence of the antifibrinolytic substance, the resulting PRP gel has longer clinical efficacy, as evidenced by the prolonged release of platelet growth factors. This beneficial effect can be standardized by forming the PRP in a centrifugation container already containing an antifibrinolytic substance i.e. the antifibrinolytic substance is present before and during the formation of PRP, resulting in the effect of the antifibrinolytic substance on the blood being enhanced. Having the antifibrinolytic substance in contact with the whole blood at a very early stage provides an end product with greater stability.

Thus, in a first aspect of the invention there is provided a centrifugation container comprising an antifibrinolytic substance (as shown in FIG. 1: X)

The antifibrinolytic substance is a substance (e.g. a compound) which inhibits fibrinolysis. Antifibrinolytic substances prevent or reduce the activation of plasminogen to form plasmin, thereby preventing or reducing blood clot degradation. Antifibrinolytic substances are typically synthetic analogues of the amino acid lysine. In one embodiment, the antifibrinolytic substance is selected from the group consisting of tranexamic acid, aminocaproic acid and aprotinin, or any combination thereof. Suitably, the antifibrinolytic substance is a small molecule e.g. with molecular weight of 500 Da or less, e.g. 400 Da or less, 300 Da or less, or 200 Da or less. In one embodiment, the antifibrinolytic substance is not an enzyme, a polypeptide or a protein. In one embodiment, the antifibrinolytic substance is tranexamic acid, aminocaproic acid or a mixture thereof. In a preferred embodiment, the antifibrinolytic substance is tranexamic acid. Suitably, the antifibrinolytic substance is 5 wt. % tranexamic acid (suitably in water). Reference to “an” antifibrinolytic substance is intended to encompass “at least one” antifibrinolytic substance, and combinations of antifibrinolytic substances are also envisaged.

In one embodiment, the centrifugation container comprises between about 5% and 25% (w/v) of antifibrinolytic substance e.g. between about 10% and about 20% (w/v). Suitably, the antifibrinolytic substance is in water e.g. water for injection. Suitably, the centrifugation container comprises between about 0.1 mL and about 1 mL of antifibrinolytic substance, such as between about 0.1 mL and about 0.6 mL, in particular between about 0.2 mL and about 0.5 mL, such as about 0.3 mL. In a preferred embodiment, the antifibrinolytic substance is tranexamic acid, and the centrifugation container comprises between about 20 mg and 200 mg of tranexamic acid, e.g. between about 50 mg and about 150 mg, e.g. about 80 mg, about 90 mg, about 100 mg, about 110 mg, about 120 mg, about 125 mg, or about 130 mg of tranexamic acid. In this embodiment, suitably the centrifugation container is a 20 mL (fill volume) centrifugation tube.

The centrifugation container of the present invention optionally comprises a density gradient medium. Reference to “a” density gradient medium is intended to encompass “at least one” density gradient medium, and combinations of density gradient media are also envisaged. Each blood and bone marrow constituent has a specific density, meaning that they can be separated from one another by gravity or by centrifugal force. A primary function of the density gradient medium is to separate blood and bone marrow components, on the basis of their density. In a centrifugation tube, particles with a density which is higher than that of density gradient medium will move below the density gradient medium, and particles with a lower density will move above the density gradient medium. The density gradient medium is preferably chemically inert to components deriving from the body, such as blood and bone marrow constituents.

Suitably, the density gradient medium is a thixotropic gel. The term “thixotropic gel” is well known in the art, and refers to a gel that becomes more fluid as a result of agitation or pressure, in particular a gel having a viscosity which decreases as a result of agitation or pressure. When used in a centrifugation container, a thixotropic gel is thick or solid under static conditions, but when subjected to centrifugal force becomes more fluid and can migrate within the tube. When the centrifugation tube also contains whole blood or bone marrow, during centrifugation the blood/bone marrow components separate into layers depending on their density (those with a higher density moving towards the bottom of the tube, and those with a lower density moving towards the top of the tube). As the thixotropic gel becomes more fluid under centrifugal force, its position in the tube (along with the other components) is reflected by its density. When centrifugation ends, the thixotropic gel regains its original thick or solid consistency, acting as a mechanical barrier between blood/bone marrow constituents having a density which is higher (these constituents will end up below the thixotropic gel) and those having a density which is lower (these constituents will end up above the thixotropic gel). The mechanical barrier provided by the gel facilitates quick and easy separation of the blood/bone marrow components, leading to greater consistency in the separation process, as human error is reduced or eliminated entirely.

The centrifugation container of the present invention can comprise a single density gradient media, or can comprise two, three, four or five density gradient media. Preferably the density gradient medium comprises, consists essentially of or consists of a thixotropic gel. In one embodiment, the density gradient medium is a thixotropic gel. Thus, in one embodiment of the invention is provided a centrifugation container comprising, consisting essentially of, or consisting of an antifibrinolytic substance and a thixotropic gel (e.g. as shown in FIG. 1: A and B). In this embodiment, the centrifugation container can comprise a single thixotropic gel, or can comprise two, three, four or five thixotropic gels.

In one embodiment, the density gradient medium (e.g. thixotropic gel) has a density between about 1.010 g/cm³ and about 1.095 g/cm³, such as about 1.010 g/cm³, about 1.015 g/cm³, about 1.020 g/cm³, about 1.025 g/cm³, about 1.030 g/cm³, about 1.035 g/cm³, about 1.040 g/cm³, about 1.045 g/cm³, about 1.050 g/cm³, about 1.055 g/cm³, about 1.060 g/cm³, about 1.065 g/cm³, about 1.070 g/cm³, about 1.075 g/cm³, about 1.080 g/cm³, about 1.085 g/cm³, about 1.090 g/cm³, or about 1.095 g/cm³. All densities of density gradient media (e.g. thixotropic gels) described herein are measured at 25° C. at atmospheric pressure.

In one embodiment, the density gradient medium (e.g. thixotropic gel) has a density between about 1.045 g/cm³ and about 1.095 g/cm³, such as between about 1.050 g/cm³ and about 1.095 g/cm³, or between about 1.055 g/cm³ and about 1.095 g/cm³. In whole blood, platelets (also known as thrombocytes) typically have a density of around 1.040 g/cm³. As such, when centrifuged with a density gradient medium with density between about 1.045 and about 1.095 g/cm³, (i.e. a density gradient medium with higher density), following centrifugation all or the majority of the platelets will reside above the layer of density gradient medium. As such, using a density gradient medium with density between about 1.045 g/cm³ and about 1.095 g/cm³ facilitates the preparation of platelet rich plasma (PRP).

In one embodiment, the density gradient medium (e.g. thixotropic gel) has a density between about 1.070 g/cm³ and about 1.090 g/cm³, such as between about 1.075 g/cm³ and about 1.090 g/cm³, about 1.080 g/cm³ and about 1.090 g/cm³, between about 1.070 g/cm³ and about 1.080 g/cm³, or between about 1.075 g/cm³ and about 1.080 g/cm³, e.g. about 1.075 g/cm³. In another embodiment, the density gradient medium (e.g. thixotropic gel) has a density between about 1.045 g/cm³ and about 1.075 g/cm³, such as between about 1.045 g/cm³ and about 1.055 g/cm³, between about 1.050 g/cm³ and about 1.070 g/cm³, or between about 1.050 g/cm³ and about 1.060 g/cm³, e.g. about 1.055 g/cm³.

In whole blood, white blood cells (leukocytes) typically have a density of between about 1.060 g/cm³ and about 1.085 g/cm³. Thus, when centrifuged with a density gradient medium (e.g. thixotropic gel) with density between about 1.045 g/cm³ and about 1.055 g/cm³ (i.e. density gradient medium with lower density), following centrifugation all or the majority of leukocytes will reside below the layer of density gradient medium. As such, using a density gradient medium with density between about 1.045 g/cm³ and about 1.055 g/cm³ facilitates the preparation of leukocyte-poor platelet rich plasma (LP-PRP).

Monocytes and lymphocytes (types of leukocytes which are also known as agranulocytes) have density of between about 1.060 g/cm³ and about 1.075 g/cm³. As such, when centrifuged with a density gradient medium (e.g. thixotropic gel) with density between about 1.070 g/cm³ and about 1.090 g/cm³ (i.e. density gradient medium with higher density), following centrifugation all or the majority of monocytes and lymphocytes will reside above the layer of density gradient media. Basophils, neutrophils and eosinophils (types of leukocytes which are also known as granulocytes) have density of between about 1.072 g/cm³ and about 1.10 g/cm³. As such, using a density gradient medium with density between about 1.070 g/cm³ and about 1.090 g/cm³ facilitates the preparation of agranulocyte-rich PRP (which is rich in monocytes and lymphocytes) while also being granulocyte poor (i.e. depleted levels of basophils, neutrophils and eosinophils). Even though enhanced in agranulocytes, this PRP is considered overall to be leukocyte-poor PRP (LP-PRP).

In one embodiment, the density gradient medium (e.g. thixotropic gel) has a density between about 1.020 g/cm³ and about 1.050 g/cm³, such as between about 1.025 g/cm³ and about 1.040 g/cm³, e.g. about 1.030 g/cm³. Essentially all cellular constituents in whole blood have a density that is greater than 1.050 g/cm³, therefore using a density gradient medium with density between about 1.020 g/cm³ and about 1.050 g/cm³ facilitates the preparation of essentially acellularized plasma.

Which density gradient medium (e.g. thixotropic gel) to use depends on the intended therapeutic application of the platelet rich plasma or bone marrow composition. Essentially acellularized plasma has utility as “convalescent plasma” which is of particular use in the treatment and/or prophylaxis of viral infections and associated conditions, as described in WO2021/198312A1. Essentially acellularized plasma may also have utility when combined with an antifibrinolytic for use in treating or preventing coagulopathy e.g. traumatic coagulopathy (Kuckelman et al., 2018). For such applications, use of a density gradient medium (e.g. thixotropic gel) with density between about 1.020 g/cm³ and about 1.050 g/cm³ is preferred. In this embodiment, suitably the thixotropic gel comprises, consists essentially of or consists of silica dimethyl silylate, a polyoxyalkylene polyol, trioctyl trimellitate, or a hydrocarbonated resin, or any combination thereof.

Leukocytes can be divided into three main populations in blood: granulocytes (65%), lymphocytes (30%) and monocytes (5%). Monocytes and lymphocytes play a positive role in tissue healing. Monocytes differentiate into macrophages in tissue, and through their phagocytic activity clear the wound of dead cells and other debris. They are also involved in the resolution of inflammation (Brancato et al., 2011). Lymphocytes can secrete large amounts of growth factors that stimulate angiogenesis and new collagen deposition by fibroblasts (Schaffer et al., 1998). Granulocytes, on the other hand are pro-inflammatory cells containing potent destructive enzymes such as peroxidases, proteases and collagenases. The release of these molecules is crucial for fighting bacterial infection in open wounds; however, it has a deleterious effect in aseptic wounds. Thus, as granulocytes are the most abundant form of leukocyte, using a leukocyte-poor PRP is advantageous in certain applications to avoid undesired inflammatory reactions. For such applications, use of a density gradient medium (e.g. thixotropic gel) with density between about 1.045 g/cm³ and about 1.055 g/cm³ is preferred. Commercially available A-CP tubes and Regen BCT tubes (both manufactured by Regen Lab) contain thixotropic gels with such densities.

However, in certain applications retaining the monocytes and lymphocytes in the PRP could have potentially beneficial effects. For such applications, use of a density gradient medium (e.g. thixotropic gel) with density between about 1.060 g/cm³ and about 1.085 g/cm³ is preferred. This density allows specific depletion of granulocytes while maintaining a potentially beneficial population of monocytes and leukocytes. The PRP produced in mononuclear cell-rich (monocytes and leukocytes being mononuclear cells), but is overall still considered to be leukocyte poor, because the total leukocyte concentration is below that of the whole blood prior to centrifugation. Commercially available RegenTHT tubes (also manufactured by Regen Lab) contain thixotropic gels within this density range.

Because of its ability to recover mononuclear cells with platelets and plasma, the centrifugation container of the present invention can also be used to process bone marrow aspirate. Like blood, bone marrow aspirate consists of a suspension of blood cells as well as stem cells, precursors and immature cells of the different cell lineages produced by the bone marrow in plasma. When bone marrow aspirate is centrifuged in a container of the invention comprising a density gradient medium (in particular a thixotropic gel), the nature of the density gradient medium can be selected (as described in detail above) in order that mature red and white blood cells as well as most immature blood cells migrate below the density gradient medium, while platelets and the mononuclear cell fraction remain above the density gradient medium and can be recovered within the plasma. The mononuclear cell fraction of bone marrow contains not only mature blood mononuclear cells but also hematopoietic progenitor cells and mesenchymal stem cells. Commercially available RegenTHT tubes have been shown to recover mesenchymal stem cells with high efficiency (>87%) which is the highest yield when compared to other systems for bone marrow processing.

The thixotropic gel is typically a large polymer complex. In one embodiment, the thixotropic gel comprises, consists essentially of or consists of an oligomer or polymer selected from the group consisting of a polyolefin hydrocarbon oligomer, a polyester gel, an acrylic resin mixture, a silica (such as silica dimethyl silylate), a PEG-silica gel, a polyoxyalkylene polyol, trioctyl trimellitate, a hydrocarbonated resin, or any combination thereof.

Suitable polyoxyalkylene polyols include polyethylene glycol trimethylolpropane ether, polypropylene glycol trimethylolpropane ether, methyloxirane polymer with oxirane, ether with 2-ethyl-2-(hydroxymethyl)-1,3-propanediol; poly(oxyethylene) trimethylolpropane ether, poly(oxypropylene)trimethylolpropane ether, trimethylol propane, ethoxylated trimethylolpropane, propxylated trimethylol propane, or any combination thereof.

The polyoxyalkylene polyol preferably comprises hydroxyl group containing groups of formula 1:

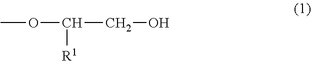

Suitably trioctyl trimellitate is tris(2-ethylhexyl) trimellitate.

Suitable hydrocarbonated resins include a cycloaliphatic hydrocarbon resin.

In one embodiment, the thixotropic gel comprises two different polymers/oligomers selected from the group consisting of a polyolefin hydrocarbon oligomer, a polyester gel, an acrylic resin mixture, an oligomeric or polymeric silica (such as silica dimethyl silylate), a PEG-silica gel, a polyoxyalkylene polyol, trioctyl trimellitate, a hydrocarbonated resin. In a preferred embodiment, the thixotropic gel comprises trioctyl trimellitate and/or a hydrocarbon resin, and in particular comprises trioctyl trimellitate and a hydrocarbon resin.

In one embodiment, the thixotropic gel comprises three different polymers/oligomers selected from the group consisting of a polyolefin hydrocarbon oligomer, a polyester gel, an acrylic resin mixture, a silica (such as silica dimethyl silylate), a PEG-silica gel, a polyoxyalkylene polyol, trioctyl trimellitate, a hydrocarbon resin.

In one embodiment, the thixotropic gel comprises four different polymers/oligomers selected from the group consisting of a polyolefin hydrocarbon oligomer, a polyester gel, an acrylic resin mixture, a silica (such as silica dimethyl silylate), a PEG-silica gel, a polyoxyalkylene polyol, trioctyl trimellitate, a hydrocarbon resin.

The thixotropic gel can contain components in addition to the oligomer or polymer. In one embodiment, in addition to the oligomer or polymer, the thixotropic gel further comprises one or more additives selected from the group consisting of tris(2-ethylhexyl)benzene-1,2,4-tricarboxylate, silicon dioxide, a silane, a dichlorodimethyl-reaction product, a monomeric silica (such as dimethyl dichlorosilane), a phenolic compound (such as tetrakis (3-(3,5-di-tert-butyl-4-hydroxyphenyl) propionate of pentaerythritol), a polyol (such as a polyalkylene polyol), a phosphite ester (such as the phosphite ester of tris(2,4-di-tert-butylphenyle)), and an azelate ester. In one embodiment, in addition to the oligomer or polymer, the thixotropic gel further comprises further comprises a phenol and/or a phosphite ester.

In one embodiment, the thixotropic gel comprises, consists essentially of or consists of silica dimethyl silylate, a polyoxyalkylene polyol, trioctyl trimellitate, or a hydrocarbonated resin, or any combination thereof.

In one embodiment, the thixotropic gel comprises trioctyl trimellitate and/or a hydrocarbon resin, and in particular comprises trioctyl trimellitate and a hydrocarbon resin. In this embodiment, the thixotropic gel optionally further comprises a phenol and/or a phosphite ester.

In one embodiment, the thixotropic gel comprises, consists essentially of, or consists of trioctyl trimellitate (in particular tris(2-ethylhexyl) trimellitate), a hydrocarbon resin (in particular a cycloaliphatic hydrocarbon resin), a monomeric silica (in particular dimethyl dichlorosilane), a polyoxyalkylene polyol, a phenolic compound and a phosphite ester (in particular the phosphite ester of tris(2,4-di-tert-butylphenyle)). In this embodiment, suitably trioctyl trimellitate is present in an amount between about 40 wt. % and about 60 wt. % (for example about 50.96 wt. %); the hydrocarbon resin present in an amount between about 30 wt. % and about 60 wt. % (for example about 43 wt. %); the silica is present in an amount between about 2 wt. % and about 10 wt. % (for example about 4.21 wt. %); the polyoxyalkylene polyol is present in an amount between about 1 wt. % and about 5 wt. % (for example about 1.73 wt. %); the phenolic compound is present in an amount between about 0 wt. % and about 1 wt. % (for example about 0.05 wt. %); and the phosphite ester is present in an amount between about 0 wt. % and about 0.06 wt. % (for example about 0.05 wt. %).

In one embodiment, the thixotropic gel comprises, consists essentially of or consists of trioctyl trimellitate (which is suitably present in an amount between about 35 wt. % and about 55 wt. %), silica (which is suitably present in an amount between about 2 wt. % and about 10 wt. %), a hydrocarbon resin (which is suitably present in an amount between about 20 wt. % and about 40 wt. %), an azelate ester (which is suitably present in an amount between about 10 wt. % and about 30 wt. %), and a phenol (which is suitably present in an amount between about 0 wt. % and about 1 wt. %).

In one embodiment, the thixotropic gel comprises trioctyl trimellitate (which is suitably present in an amount of about 50.96 wt. %), silica (which is suitably present in an amount of about 4.21 wt. %), hydrocarbon resin (which is suitably present in an amount of about 43 wt. %), an azelate ester (which is suitably present in an amount of about 15.82 wt. %), and a phenol (which is suitably present in an amount of about 0.05 wt. %).

In one embodiment, the thixotropic gel comprises, consists or consists essentially of trioctyl trimellitate, a monomeric silica (in particular dimethyl dicholorsilane), a hydrocarbon resin (in particular a cycloaliphatic hydrocarbon resin), a phenol (in particular Tetrakis (3-(3,5-di-tert-butyl-4-hydroxyphenyl) propionate of pentaerythritol) and a phosphite ester (in particular tris (2,4-di-tert-butylphenyl)phosphite).

In one embodiment, the thixotropic gel comprises, consists essentially of or consists of trioctyl trimellitate in an amount between about 40 wt. % and about 60 wt. %, a silica in an amount between about 2 wt. % and about 10 wt. %, a hydrocarbon resin in an amount between about 30 wt. % and about 60 wt. %; a phenol in the range of between about 0 wt. % and about 1 wt. %, and a phosphite ester in an amount between about 0 wt. % and about 0.06 wt. %.

In one embodiment, the thixotropic gel comprises, consists essentially of or consists of trioctyl trimellitate in an amount of about 52.26 wt. %, a silica in an amount of about 7.99 wt. %, a hydrocarbon resin in an amount of about 39.65 wt. %, a phenol in an amount of about 0.05 wt. %, and a phosphite ester in an amount of about 0.05 wt. %.

The density gradient medium (e.g. thixotropic gel), is present in the centrifugation container in an amount which is sufficient to form a robust layer between blood/bone marrow constituents, thereby facilitating straightforward and accurate separation of the blood/bone marrow constituents. In one embodiment, the centrifugation container of the invention comprises between about 0.1 mL and about 10 mL of (total) density gradient medium (e.g. thixotropic gel), in particular between about 0.1 mL and about 0.6 mL, such as about 0.3 mL. In one embodiment, the centrifugation container of the invention comprises between about 0.5 g and about 10 g of (total) density gradient medium (e.g. thixotropic gel), in particular between about 1 g and about 5 g, such as about 3 g.

In one embodiment, the density gradient medium (e.g. thixotropic gel) is insoluble in water. In one embodiment, the density gradient medium (e.g. thixotropic gel) is partially soluble in acetone. In one embodiment, the density gradient medium (e.g. thixotropic gel) is easily soluble in hexane. In one embodiment, the density gradient medium (e.g. thixotropic gel) has a viscosity of between about 400 Pa·s and about 700 Pa·s at 15° C., such as between about 100 Pa·s and about 250 Pa·s at 25° C., between about 30 Pa·s and about 100 Pa·s at 45° C., or between about 10 Pa·s and about 80 Pa·s at 65° C.

In one embodiment, the ratio of antifibrinolytic substance to density gradient medium (v/v) is between about 1:5 and about 1:15, such as between about 1:8 and about 1:12; and in particular is about 1:10.

The antifibrinolytic substance may be located above or below the layer of density gradient medium (e.g. thixotropic gel) in the centrifugation container. Thus, in one embodiment, the antifibrinolytic substance is located above the density gradient medium, or at a more proximal end of the container than the density gradient medium (see for example FIG. 1: A). In another embodiment, the antifibrinolytic substance is located below the density gradient medium, or at a more distal end of the container than the density gradient medium (see for example FIG. 1: B). The proximal end of the container is the end via which the material to be centrifuged (e.g. blood or bone marrow) is collected. The distal end of the container is the end which is opposite the proximal end of the container. In a preferred embodiment, the antifibrinolytic substance is located below the density gradient medium.

The centrifugation container of the present invention optionally further comprises an anticoagulant. In one embodiment, the anticoagulant is selected from the group consisting of sodium citrate, acid citrate dextrose (ACD), modified ACD, heparin or a salt thereof, ethylenediaminetetraacetic acid (EDTA) or a salt thereof, an iodo acetate salt, an oxalate salt, and a fluoride salt. The anticoagulant can be prepared as a solution in water, and can be wet sprayed on an inner wall of the centrifugation container. Alternatively, the anticoagulant can be a lyophilised material which is dry sprayed on an inner wall of the centrifugation container. In a preferred embodiment, the anticoagulant is sodium citrate. Suitably, the sodium citrate is a solution of sodium citrate in water. Sodium citrate includes hydrates thereof, e.g. sodium citrate dihydrate.

In one embodiment, the centrifugation container comprises between about 0.5% and about 10% (w/v) of anticoagulant e.g. between about 1% and about 5%, e.g. about 2.5% or about 4% (w/v). Suitably, the anticoagulant is in water e.g. water for injection. In one embodiment, the anticoagulant is present at a concentration of between about 0.05 M and about 0.15 M, such as between about 0.08 M and about 0.14 M, such as about 0.1 M.

Suitably, the anticoagulant is located at a proximal end of the centrifugation container, in order that it comes into immediate contact the whole blood or bone marrow, following collection.

Where the centrifugation container also contains a density gradient medium (e.g. a thixotropic gel), suitably the anticoagulant is located above the density gradient medium, or at a more proximal end of the container than the density gradient medium. In one embodiment, both the anticoagulant and antifibrinolytic substance are located above the density gradient medium, or at a more proximal end of the container than the density gradient medium (see for example FIG. 1: C), where a mixture of anticoagulant and antifibrinolytic substance is formed i.e. they do not form separate layers within the centrifugation container. In another embodiment, the anticoagulant is located above the density gradient medium, or at a more proximal end of the container than the density gradient media; and the antifibrinolytic substance is located below the density gradient medium, or at a more distal end of the container than the density gradient medium (see for example FIG. 1: D).

The anticoagulant is used to prevent coagulation during PRP preparation and to maintain the PRP in a liquid state. In centrifugation containers of the invention containing an antifibrinolytic substance but not containing an anticoagulant, a plasma clot will typically be obtained following centrifugation.

Commercially available RegenATS tubes contain a thixotropic gel but no anticoagulant. Following collection of whole blood and centrifugation, a plasma clot is formed which resides above the thixotropic gel. Serum can be extracted from this clot (known as autologous thrombin serum (ATS)) which contains among other things, the enzyme thrombin in its activated form. As discussed above, thrombin is the enzyme that converts soluble plasma fibrinogen in fibrin that polymerizes and forms clots. When ATS is added to a solution of PRP and anticoagulant (and optional coagulation activator), there are enough units of activated thrombin in ATS to trigger coagulation to form a platelet gel, fibrin glue, fibrin membrane or fibrin clot.

In embodiments of the centrifugation container of the invention containing an antifibrinolytic substance but not an anticoagulant (see for example FIG. 1: X), it is expected that the antifibrinolytic substance will provide enhanced mechanical stability to the resulting plasma clot, and hence will produce serum with enhanced properties.

The centrifugation container of the present invention optionally further comprises a coagulation activator. A coagulation activator is an agent, for example a compound or an enzyme, that is able to trigger or activate coagulation of plasma and platelets aggregation, forming a clot, which may have the consistency of a gel. Reference to “a” coagulation activator is intended to encompass “at least one” coagulation activator, and combinations of coagulation activators are also envisaged.

Typically, the coagulation activator is a compound or moiety which is a thrombin activator and/or a fibrinogen activator. In one embodiment, the coagulation activator is a compound selected from the group consisting of a calcium salt and thrombin. Suitably the calcium salt is selected from the group consisting of calcium gluconate, calcium carbonate, calcium sulphate, calcium saccharate and calcium chloride; or any combination thereof, and in particular is calcium gluconate.

In one embodiment, a coagulation activator is combined with the composition formed after the whole blood or bone marrow has been collected in the centrifugation container of the invention, and centrifuged (i.e. the coagulation activator is not present in the centrifugation container of the invention prior to centrifugation). Suitably, the coagulation activator in this embodiment is autologous thrombin serum (ATS—as described above) or calcium gluconate.

In one embodiment, the coagulation activator is a calcium salt (in particular calcium gluconate) in a solution of water at between about 1 wt. % and about 20 w. % e.g. between about 5 wt. % and about 15 wt. % such as about 10 wt. %.

The calcium salt may comprise a mixture of calcium salt e.g. a combination of calcium gluconate and calcium saccharate.

In one embodiment, the centrifugation container of the invention comprises between about 0.1 mL and about 1.0 mL of coagulation activator, in particular between about 0.1 mL and about 0.6 mL, such as about 0.3 mL.

In one embodiment, the coagulation activator is located above the antifibrinolytic substance, or at a more proximal end of the container than the antifibrinolytic substance. In another embodiment, the coagulation activator is located below the antifibrinolytic substance, or at a more distal end of the container than the antifibrinolytic substance. In one embodiment, the centrifugation container of the invention comprises a mixture of coagulation activator and antifibrinolytic substance.

When the centrifugation container further comprises a density gradient medium (e.g. a thixotropic gel) the coagulation activator can reside above or below the density gradient medium. Thus, in one embodiment, the coagulation activator is located above the density gradient medium, or at a more proximal end of the container than the density gradient medium. In another embodiment, the coagulation activator is located below the density gradient medium, or at a more distal end of the container than the density gradient medium. Suitably, both the coagulation activator and the antifibrinolytic substance are located below the density gradient medium, or at a more distal end of the container than the density gradient medium. (see for example FIG. 1: F), where a mixture of coagulation activator and antifibrinolytic substance is formed i.e. they do not form separate layers within the centrifugation container.

In one embodiment, the ratio of coagulation activator to antifibrinolytic substance (v/v) is between about 10:1 and about 1:10, such as between about 5:1 and about 1:5; and in particular is about 1:1.

In one embodiment, the ratio of coagulation activator to density gradient medium (e.g. thixotropic gel) (v/v) is between about 1:20 and about 1:5, such as between about 1:15 and about 1:8; and in particular is about 1:10.

The centrifugation container of the present invention optionally further comprises a structural biomaterial. Reference to “a” structural biomaterial is intended to encompass “at least one” structural biomaterial, and combinations of structural biomaterials are also envisaged. A primary function of the structural biomaterial is to create an enhanced scaffold within the fibrin clot which allows for longer residence time of the PRP and associated enhanced release of growth factors. The structural biomaterial may itself bind extracellular matrix proteins and induce migration of cells for tissue repair.

Typical structural biomaterials include a glycosaminoglycan, a silk protein, fibroin, collagen, polysaccharide or a polylactide, or any combination thereof. In one embodiment, the structural biomaterial is a glycosaminoglycan (which can be derivatised), such as selected from the group consisting of chondroitin, chondroitin sulfate, dermatan, dermatan sulfate, heparin, heparan sulfate, heparosan, hyaluronan and hyaluronic acid. In one embodiment, the structural biomaterial is a polysaccharide (which can be derivatised), such as selected from the group consisting of sucrose, lactulose, lactose, maltose, trehalose, cellobiose, mannobiose, chitobiose, chitosan and cellulose.

A glycosaminoglycan can be derivatised with one or more side chains, e.g. side chains comprising a polyalkylene glycol-containing residue.

In a preferred embodiment, the structural biomaterial is a glycosaminoglycan such as heparosan or hyaluronic acid, and in particular is hyaluronic acid.

The present inventors have discovered that including a structural biomaterial such as hyaluronic acid in the centrifugation tube further stabilizes the end product by hindering uncontrolled release of growth factors. The hyaluronic acid also enhances/stimulates the presence of growth factors at injury site. It is believed that the PRP, antifibrinolytic substance and structural biomaterial (e.g. hyaluronic acid) have a synergistic action. The combination of PRP, antifibrinolytic substance, coagulation activator (e.g. calcium gluconate) and structural biomaterial (e.g. hyaluronic acid) is particularly beneficial, as shown in Example 5.

The biological effects of hyaluronic acid are related to its molecular weight. In one embodiment, the hyaluronic acid is low molecular weight hyaluronic acid (LMW-HA), with molecular weight between about 400 kDa and about 1,000 kDa. In another embodiment, the hyaluronic acid is middle molecular weight hyaluronic acid, with molecular weight between about 1,000 kDa and about 1,800 kDa, such as between about 1,400 kDa and about 1,600 kDa, e.g. about 1,500 kDa. In another embodiment, the hyaluronic acid is high molecular weight hyaluronic acid (HMW-HA), with molecular weight greater than 1,800 kDa. Preferably, the hyaluronic acid in the centrifugation container of the invention has molecular weight between about 1,000 kDa and about 1,800 kDa, such as between about 1,400 kDa and about 1,600 kDa, e.g. about 1,500 kDa.

A mixture of hyaluronic acids with different molecular weights may also be used. Thus, in one embodiment, the hyaluronic acid is a mixture of hyaluronic acids comprising:
a low molecular weight hyaluronic acid (LMW-HA), with molecular weight between about 400 kDa and about 1,000 kDa; and/or
a middle molecular weight hyaluronic acid, with molecular weight between about 1,000 kDa and about 1,800 kDa, such as between about 1,400 kDa and about 1,600 kDa, e.g. about 1,500 kDa; and/or
a high molecular weight hyaluronic acid (HMW-HA), with molecular weight greater than 1, 800 kDa.

In one embodiment, the hyaluronic acid is cross-linked. In another embodiment, the hyaluronic acid is linear.

The hyaluronic acid is preferably added as a solution in water, wherein the concentration of hyaluronic acid in the solution (added to the container) is between about 1 wt. % and about 5 wt. %, such as between about 1.8% and about 2.2 wt. %. In one embodiment, the centrifugation container comprises between about 1.0 mL and about 5.0 mL of the hyaluronic acid, in particular between about 1.5 mL and about 3 mL, such as about 2 mL. In this embodiment, suitably the fill volume of the centrifugation container is 10 mL or 15 mL, in particular 15 mL.

In one embodiment, the structural biomaterial (e.g. hyaluronic acid) is located above the antifibrinolytic substance, or at a more proximal end of the container than the antifibrinolytic substance. In another embodiment, the structural biomaterial (e.g. hyaluronic acid) is located below the antifibrinolytic substance, or at a more distal end of the container than the antifibrinolytic substance. In another embodiment, the centrifugation container comprises a mixture of structural biomaterial and antifibrinolytic substance.

When the centrifugation container further comprises a density gradient medium (e.g. a thixotropic gel) the structural biomaterial (e.g. hyaluronic acid) can reside above or below the density gradient medium. Thus, in one embodiment, the structural biomaterial is located above the density gradient medium, or at a more proximal end of the container than the density gradient medium. In another embodiment, the structural biomaterial is located below the density gradient medium, or at a more distal end of the container than the density gradient medium. Suitably, both the structural biomaterial and the antifibrinolytic substance are located below the density gradient medium, or at a more distal end of the container than the density gradient medium, where a mixture of structural biomaterial and antifibrinolytic substance is formed i.e. they do not form separate layers within the centrifugation container.

In one embodiment, the ratio of structural biomaterial (e.g. hyaluronic acid) to antifibrinolytic substance (v/v) is between about 10:1 and about 500:1, such as between about 5:1 and about 250:1; and in particular is about 15:1.

In one embodiment, the ratio of structural biomaterial (e.g. hyaluronic acid) to density gradient medium (v/v) is between about 1:10 and about 50:1, such as between about 1:5 and about 10:1; between about 1:1 and about 1:2, and in particular is about 1:1.6.

The centrifugation container may contain a further therapeutic agent. Reference to “a” therapeutic agent is intended to encompass “at least one” further therapeutic agent, and combinations of therapeutic agents are also envisaged. In one embodiment, the further therapeutic agent is selected from the group consisting of a steroid, a corticosteroid, a glucocorticosteroid, a non-steroidal anti-inflammatory drug (NSAID), kartogenin, an anaesthetic, an antibacterial compound, an antibiotic, an antifungal compound, an antiparasitic compound, an enzyme, an enzyme inhibitor, a glycoprotein, a growth factor, a hormone, an antiviral compound, an analgesic, an opioid, a saponin, a haemoglobin, tetrahydrocannabinol (THC), cannabidiol (CBD), an anti-angiogenetic agent, anti-melanogenetic agent, an immunomodulator, an immunoglobulin, a mineral, a neuroleptic, a protein, a peptide, a lipoprotein, a tumouricidal compound, a tumourstatic compound, a toxin, a vitamin (such as vitamin A, vitamin E, vitamin B, vitamin C, vitamin D; or a derivative thereof) or a wrinkle filler, or any combination thereof. In a preferred embodiment, the further therapeutic agent is selected from the group consisting of a corticosteroid, an NSAID, kartogenin, a haemoglobin, an anaesthetic, an analgesic, an opioid, a saponin and THC, or a combination thereof.

When the centrifugation container further comprises a density gradient medium (e.g. a thixotropic gel) the further therapeutic agent preferably resides below the density gradient medium. Thus, in one embodiment, the further therapeutic agent is located below the density gradient medium, or at a more distal end of the container than the density gradient medium. Suitably, both the further therapeutic agent and the antifibrinolytic substance are located below the density gradient medium, or at a more distal end of the container than the density gradient medium, where a mixture of further therapeutic agent and antifibrinolytic substance is formed i.e. they do not form separate layers within the centrifugation container.

Suitable NSAIDs include a molecule derived from propionic acid (such as a prostaglandin(s) inhibitor) and a COX inhibitor (such as a COX1 and/or COX2 inhibitor, for example ketorolac (2-amino-2-(hydroxymethyl)-1,3-propanediol(+)-5-benzoyl-2,3-dihydro-1H-pyrrolizine-1-carboxylic acid; CAS n° 74103-6-3 66635-83-4).

Suitable anaesthetics include those comprising an amino-amide moiety, such as bupivacaine ((RS)-1-butyl-N-(2,6-dimethylphenyl) piperidine-2-carboxamide; Pubchem n°2474).

Suitable analgesics include acetaminophen.

Suitably, the haemoglobin is more oxygenating than human haemoglobin, preferably Hg from worms, preferably Hg from Arenicola marina (lugworms), whose haemoglobin is 40 times more oxygenating than human Hg.

Suitable opioids include oxycodone, hydrocodone, morphine, and methadone.

Suitable saponins include ginsenosides.

In one embodiment, the centrifugation container of the present invention further comprises an anaesthetic (in particular bupivacaine) and an NSAID (in particular ketorolac). In one embodiment, the centrifugation container of the present invention further comprises an anaesthetic (in particular bupivacaine) and an analgesic (in particular acetaminophen). In one embodiment, the centrifugation container of the present invention further comprises an anaesthetic (in particular bupivacaine), an NSAID (in particular Ketorolac) and an analgesic (in particular acetaminophen).

In one embodiment, the centrifugation container of the present invention further comprises kartogenin.

The centrifugation container of the present invention can further comprise a cell extract. Reference to “a” cell extract is intended to encompass “at least one” cell extract, and combinations of cell extracts are also envisaged. In one embodiment, the cell extract (which is suitably autologous) is selected from an extract of keratinocytes, bone marrow, fibroblasts, periosteum or corneal cells, melanocytes and Langerhans cell, fat cells, muscle cells such as myoblasts and satellite cells, osteoblasts, chondrocytes, umbilical cord cells, stem cells, mesenchymal stem cells (MSCs), preadipocytes, pre-endothelial cells, Schwann cells or Achilles tendon cells, or any combination thereof. Suitable processes for obtaining such cell extracts are described in WO2008/023026A1, which is incorporated by reference herein in its entirety.

In one embodiment, the centrifugation container does not contain phosphate buffered saline (PBS).

In one embodiment is provided a centrifugation container comprising, consisting essentially of or consisting of a composition, said composition consisting essentially of or consisting of:
an antifibrinolytic substance;
optionally a density gradient medium; and/or
optionally an anticoagulant; and/or
optionally a structural biomaterial.

Suitable antifibrinolytic substances, density gradient media, anticoagulants and structural biomaterials are described hereinabove.

In one embodiment is provided a centrifugation container, comprising, consisting essentially of or consisting of an antifibrinolytic substance.

In one embodiment is provided a centrifugation container, comprising, consisting essentially of or consisting of an antifibrinolytic substance and a density gradient medium. In one embodiment, the antifibrinolytic substance is located above the density gradient medium, or at a more proximal end of the container than the density gradient medium (see for example FIG. 1: A). In another embodiment, the antifibrinolytic substance is located below the density gradient medium, or at a more distal end of the container than the density gradient medium (see for example FIG. 1: B).

In one embodiment is provided a centrifugation container, comprising, consisting essentially of or consisting of an antifibrinolytic substance and an anticoagulant.

In one embodiment is provided a centrifugation container, comprising, consisting essentially of or consisting of an antifibrinolytic substance, a density gradient medium and an anticoagulant. In one embodiment, the anticoagulant is located above the antifibrinolytic substance, or at a more proximal end of the container than the antifibrinolytic substance. In another embodiment, the anticoagulant is located above the density gradient medium, or at a more proximal end of the container than the density gradient medium. In another embodiment, both the anticoagulant and antifibrinolytic substance (a mixture thereof) are located above the density gradient medium, or at a more proximal end of the container than the density gradient medium (see for example FIG. 1: C). In another embodiment, the anticoagulant is located above the density gradient medium, or at a more proximal end of the container than the density gradient medium, and density gradient medium is located above the antifibrinolytic substance or at a more proximal end of the container than the fibrinolytic material (see for example FIG. 1: D).

In one embodiment is provided a centrifugation container comprising, consisting essentially of or consisting of an antifibrinolytic substance, a density gradient medium and a coagulation activator. In one embodiment, the antifibrinolytic substance is located above the density gradient medium, or at a more proximal end of the container than the density gradient medium, and the density gradient medium is located above the coagulation activator or at a more proximal end of the container than the coagulation activator (see for example FIG. 1: E). In another embodiment, both the antifibrinolytic substance and the coagulation activator (a mixture thereof) are located below the density gradient medium, or at a more distal end of the container than the density gradient medium (see for Example FIG. 1: F).

In one embodiment is provided a centrifugation container comprising, consisting essentially of or consisting of an antifibrinolytic substance, a density gradient medium, an anticoagulant and a coagulation activator. In one embodiment, both the antifibrinolytic substance and the anticoagulant (a mixture thereof) are located above the density gradient medium, or at a more proximal end of the container than the density gradient medium, and the density gradient medium is located above the coagulation activator or at a more proximal end of the container than the coagulation activator (see for example FIG. 1: G). In one embodiment, the anticoagulant is located above the density gradient medium, or at a more proximal end of the container than the density gradient medium, and the density gradient medium is located above both the antifibrinolytic substance and the coagulation activator (a mixture thereof) or at a more proximal end of the container than both the antifibrinolytic substance and the coagulation activator (a mixture thereof) (see for example FIG. 1: H).

In one embodiment is provided a centrifugation container comprising, consisting essentially of or consisting of an antifibrinolytic substance, a density gradient medium and a structural biomaterial (such as hyaluronic acid).

In one embodiment is provided a centrifugation container comprising, consisting essentially of or consisting of an antifibrinolytic substance, a density gradient medium, an anticoagulant and a structural biomaterial (such as hyaluronic acid).

In one embodiment is provided a centrifugation container comprising, consisting essentially of or consisting of an antifibrinolytic substance, a density gradient medium, an anticoagulant, a structural biomaterial (such as hyaluronic acid), and a coagulation activator. In one embodiment, both the antifibrinolytic substance and the anticoagulant (a mixture thereof) are located above the density gradient medium, or at a more proximal end of the container than the density gradient medium, and the density gradient medium is located above the coagulation activator or at a more proximal end of the container than the coagulation activator, and the coagulation activator is located above the structural biomaterial or at a more proximal end of the container than the structural biomaterial (see for example FIG. 1: I). In one embodiment, both the antifibrinolytic substance and the anticoagulant (a mixture thereof) are located above the density gradient medium, or at a more proximal end of the container than the density gradient media, and the density gradient medium is located above the structural biomaterial or at a more proximal end of the container than the structural biomaterial, and the structural biomaterial is located above the coagulation activator or at a more proximal end of the container than the coagulation activator (see for example FIG. 1: J). In one embodiment, both the antifibrinolytic substance and the anticoagulant (a mixture thereof) are located above the density gradient medium, or at a more proximal end of the container than the density gradient media, and the density gradient medium is located above a mixture of the structural biomaterial and the coagulation activator, or at a more proximal end of the container than a mixture of the structural biomaterial and the coagulation activator (see for example FIG. 1: J-1). In one embodiment, the anticoagulant is located above the density gradient medium, or at a more proximal end of the container than the density gradient medium, and the density gradient medium is located above a mixture of the antifibrinolytic substance and the coagulation activator, or at a more proximal end of the container than the a mixture of the antifibrinolytic substance and the coagulation activator, and a mixture of the antifibrinolytic substance and the coagulation activator is located above the structural biomaterial or at a more proximal end of the container than the structural biomaterial (see for example FIG. 1: K). In one embodiment, the anticoagulant is located above the density gradient medium, or at a more proximal end of the container than the density gradient medium, and the density gradient medium is located above the antifibrinolytic substance, or at a more proximal end of the container than the antifibrinolytic substance, and the antifibrinolytic substance is located above the structural biomaterial or at a more proximal end of the container than the structural biomaterial, and the structural biomaterial is located above the coagulation activator, or at a more proximal end of the container than the structural biomaterial (see for example FIG. 1: L). In one embodiment, the anticoagulant is located above the density gradient medium, or at a more proximal end of the container than the density gradient medium, and the density gradient medium is located above the coagulation activator, or at a more proximal end of the container than the coagulation activator, and the coagulation activator is located above the structural biomaterial or at a more proximal end of the container than the structural biomaterial, and the structural biomaterial is located above the antifibrinolytic substance, or at a more proximal end of the container than the antifibrinolytic substance (see for example FIG. 1: M). In one embodiment, the anticoagulant is located above the density gradient medium, or at a more proximal end of the container than the density gradient medium, and the density gradient medium is located above the structural biomaterial, or at a more proximal end of the container than the structural biomaterial, and the structural biomaterial is located above a mixture of the antifibrinolytic substance and the coagulation activator, or at a more proximal end of the container than the mixture of the antifibrinolytic substance and the coagulation activator (see for example FIG. 1: N). In one embodiment, the anticoagulant is located above the density gradient medium, or at a more proximal end of the container than the density gradient medium, and the density gradient medium is located above a mixture of the structural biomaterial, the antifibrinolytic substance and the coagulation activator, or at a more proximal end of the container than a mixture of the structural biomaterial, the antifibrinolytic substance and the coagulation activator (see for example FIG. 1: O).

In these embodiments, the antifibrinolytic substance is suitably tranexamic acid, the density gradient media is suitably a thixotropic gel, the anticoagulant is suitably sodium citrate, and the coagulation activator is suitably calcium gluconate.

In all of these embodiments, the centrifugation container can further comprise an additional therapeutic agent e.g. selected from the group consisting of a corticosteroid, a haemoglobin, an anaesthetic, an analgesic, an opioid, a saponin and THC, or a combination thereof.

In one embodiment is provided a centrifugation container comprising, consisting essentially of or consisting of a corticosteroid, an anaesthetic (e.g. bupivacaine), a non-steroidal anti-inflammatory drug (e.g. Ketorolac), an analgesic (e.g. acetaminophen), kartogenin and/or haemoglobin, or a combination thereof. In a preferred embodiment, the centrifugation container comprises, consists essentially of or consists of kartogenin. In this embodiment, the centrifugation container can further comprise: an antifibrinolytic substance (e.g. tranexamic acid); and/or a density gradient medium (e.g. a thixotropic gel); and/or an anticoagulant (e.g. sodium citrate); and/or a coagulation activator (e.g. sodium gluconate); and/or a further therapeutic agent. Embodiments and preferences described herein above in respect of the antifibrinolytic substance, anticoagulant, density gradient medium, coagulation activator and further therapeutic agent apply equally to these embodiments.

As mentioned above, when an antifibrinolytic substance is mixed with liquid PRP (preferably as the PRP is formed, i.e. by being present in the centrifugation container), the stability of a resulting platelet rich plasma gel/clot is enhanced, and hence the efficacy of the PRP is enhanced. In embodiments where the centrifugation container also contains a further therapeutic agent, a similar effect is observed as the therapeutic agent is also retained in the stabilized gel/clot, and thereby displays enhanced and sustained/prolonged activity.

The centrifugation container is suitably a centrifugation tube or a centrifugation syringe.

In one embodiment, the centrifugation container is made of glass, for example borosilicate glass (in particular type 1 borosilicate which is pharma injectable). In another embodiment, the container is made of one or more materials selected from the group consisting of silicone, glass, modified polyamide (MPA), polyethylene terephthalate, synthetic copolymers, ceramic and glass-ceramics, bioartificial blends of natural and synthetic materials. It should be understood that “comprises” in this context means that the material of the centrifugation container is composed of the material, rather than simply containing/holding the material (such as it does for the antifibrinolytic substance etc). Suitably, the centrifugation container is depyrogenated.

The centrifugation container can be coated with one or more substances, for example silicone and/or polypropylene. The outer wall and/or the inner wall of the container can be coated. Suitably, the inner wall of the centrifugation container is coated.

In a preferred embodiment, the centrifugation container is made from glass (in particular borosilicate glass), and also contains modified polyamide (MPA) or polyethyleneterephthalate (PET). Suitably, an interior wall is coated with polypropylene.

In one embodiment, the centrifugation container is closed to the atmosphere. In one embodiment, the centrifugation container is under vacuum. Having the container under vacuum allows a pre-determined amount of fluid (e.g. whole blood or bone marrow) to be aspirated into the container. The centrifugation container can be closed using any suitable means. Suitably, the centrifugation container (in particular a centrifugation tube) includes a plastic stopper, for example a stopper comprised of butyl rubber (for example bromobutyl rubber) or halo butyl rubber having a hardness of 40-60 Shore A. The centrifugation container preferably has a shelf life with stable vacuum of 18-24 months.

The centrifugation container has a proximal end and a distal end. The proximal end of the container is the end via which the material to be centrifuged (e.g. blood or bone marrow) is collected. The distal end of the container is the end which is opposite the proximal end of the container.

In one embodiment, the centrifugation container (preferably a centrifugation tube), is a 10 mL, 15 mL or 20 mL container (fill volume). The container may have a length (as measured between the aperture though which the whole blood or bone marrow is collected and the base of the container) of approximately 130 mm (for example 131.6 mm), and a width (as measured between opposed surfaces of the container) of approximately 15.5 mm. The container may have a length (as measured between the aperture though which the whole blood or bone marrow is collected and the base of the container) of approximately 130 mm (for example 129.5 mm), and a width (as measured between opposed surfaces of the container) of approximately 21.4 mm.

In one embodiment, the centrifugation container (preferably a centrifugation tube), is a 20 mL container (fill volume) comprising a density gradient medium (e.g. a thixotropic gel) and an anticoagulant (e.g. sodium citrate), wherein the volume ratio of density gradient medium to anticoagulant is between about 1:1 and about 4:1. Suitably, approximately 4 mL or approximately 6 mL of density gradient medium is present in the container.

In a further aspect of the invention is provided a process for preparing a medical composition, comprising the steps of:
(a) centrifuging whole blood or bone marrow in a centrifugation container as described hereinabove; and then
(b) collecting the medical composition.

In a further aspect is provided a medical composition obtained according to this process.

In one embodiment, the process comprises the steps of:
(a) centrifuging whole blood in a centrifugation container comprising an antifibrinolytic substance; and then
(b) collecting the medical composition;
wherein the medical composition is platelet rich fibrin (PRF);
wherein in step (a) the centrifugation container does not contain an anticoagulant.

In one embodiment, the process comprises the steps of:
(a) centrifuging whole blood in a centrifugation container comprising an antifibrinolytic substance and an anticoagulant; and then
(b) collecting the medical composition;
wherein the medical composition is platelet rich plasma (PRP).

In both of the above embodiments (for preparing PRF or PRP) in step (a) the centrifugation container can further comprise a coagulation activator (as described hereinabove, such as calcium gluconate) and/or a structural biomaterial (as described hereinabove, such as a hyaluronic acid). Preferably, the centrifugation container comprises a density gradient medium (e.g. a thixotropic gel). When the centrifugation container contains a density gradient medium, in step (b) the medical composition is collected from above the density gradient medium, and the material above the density gradient medium is suitably homogenized before being collected. The reason for this homogenization is that when a sufficient centrifugal force is used, the majority or all of the platelets are concentrated on the upper surface of the density gradient medium, forming a thin sediment. Homogenization of the platelets e.g. by gentle inversion of the container resuspends the platelets in the plasma.

In all of the above processes, suitably the single centrifugation in step (a) is the only centrifugation step in the process. In one embodiment, the centrifugation in step (a) is performed at a force of between about 1500 g and about 2000 g. In one embodiment, the centrifugation in step (a) is performed for a period of time between about 3 minutes and about 40 minutes. When whole blood is centrifuged, the centrifugation at least separates the red blood cells from the remaining plasma components. Further separation of the plasma components may occur, depending on the centrifugation conditions and whether a density gradient medium (such as a thixotropic gel) is present in the centrifugation container. When bone marrow is centrifuged, the centrifugation at least separates a fraction depleted in stem cells from a fraction which is enriched in stem cells.

In one embodiment, the centrifugation container of the present invention has been sterilized. Preferably, the centrifugation container is steam sterilized, at a temperature greater than 100° C., such as greater than 110° C., greater than 115° C., greater than 120° C. or greater than 121° C.

The medical composition as described herein (e.g. prepared using a centrifugation container according to the present invention, and/or prepared according to a process of the present invention), is of use in therapy.

In one embodiment is provided a medical composition comprising, consisting essentially of or consisting of:
platelet rich plasma, platelet rich fibrin, bone marrow concentrate or a combination thereof and;
an antifibrinolytic substance.

In one embodiment, is provided a platelet rich plasma (PRP) composition comprising, consisting essentially of or consisting of:
platelet rich plasma; and
an antifibrinolytic substance.

In one embodiment, is provided a platelet rich fibrin (PRF) composition comprising, consisting essentially of or consisting of:
platelet rich fibrin; and
an antifibrinolytic substance.

In all of these embodiments, the antifibrinolytic substance is as described hereinabove. When the antifibrinolytic substance is tranexamic acid, suitably it is present in the composition at a concentration of between about 10 mg/mL and about 30 mg/mL, such as between about 15 mg/mL and about 25 mg/mL e.g. about 20 mg/mL. The present inventors have found these concentrations to be optimal. See Example 1, where 20 mg/mL tranexamic acid was found to have an even more beneficial effect than 10 mg/mL. However, the present inventors believe that higher concentrations of tranexamic acid could potentially have a detrimental effect, due to the accumulation of fibrin. The preparation of medical compositions according to the invention are described in Examples 6 and 7.

In one embodiment the PRP or PRF composition further comprises a coagulation activator (as described hereinabove, in particular calcium gluconate). In one embodiment, the PRP or PRF composition further comprises a structural biomaterial (as described hereinabove, in particular heparosan or hyaluronic acid, in particular hyaluronic acid). In one embodiment, the PRP or PRF composition further comprises a further therapeutic agent selected from the group consisting of a corticosteroid, an NSAID (in particular ketorolac), kartogenin, a haemoglobin, an anaesthetic (as described herein above, in particular bupivacaine), and an analgesic (in particular acetaminophen), or any combination thereof.

In a preferred embodiment is provided a medical composition comprising, consisting essentially of or consisting of:
platelet rich plasma, platelet rich fibrin, bone marrow concentrate or a combination thereof and;
an antifibrinolytic substance;
a coagulation activator; and
a structural biomaterial.

The antifibrinolytic substance is as described herein above, and in particular is tranexamic acid. The coagulation activator is as described hereinabove, in particular is calcium gluconate. The structural biomaterial is as described hereinabove, and in particular heparosan or hyaluronic acid, and is preferably hyaluronic acid. In this embodiment, the medical composition optionally comprises a further therapeutic agent selected from the group consisting of a corticosteroid, an NSAID (in particular ketorolac), kartogenin, a haemoglobin, an anaesthetic (as described herein above, in particular bupivacaine), and an analgesic (in particular acetaminophen), or any combination thereof.

In one embodiment, is provided the medical composition as described herein, for use in treating or preventing a joint disorder or condition. Suitably, the joint disorder or condition is selected from the group consisting of arthritis, gout, fibromyalgia, lupus, polymyalgia and rheumatica. Arthritis includes osteoarthritis, rheumatoid arthritis, ankylosing spondylitis, cervical spondylitis, psoriatic arthritis, enteropathic arthritis, oligoarthritis, polyarthritis and secondary arthritis. The medical composition as described herein is of particular use in a knee arthroscopy procedure.

In one embodiment, is provided the medical composition as described herein, for use in treating a wound. In one embodiment, is provided the medical composition as described herein, for use in regenerating tissue (e.g. damaged tissue).

In one embodiment, is provided the medical composition as described herein, for use in post-surgical accelerated healing, the prevention of post-operative infections and/or the prevention of bleeding.

In one embodiment, is provided the medical composition as described herein, for use in treating or preventing melasma. In one embodiment, is provided the cosmetic use of a medical composition as described herein, for treating or preventing melasma.

In one embodiment, is provided the medical composition as described herein, for use as an anti-aging agent. In one embodiment, is provided the cosmetic use of a medical composition as described herein, for anti-aging.

In one embodiment, is provided the medical composition as described herein, for use in organ transplantation.

In one embodiment, is provided the medical composition as described herein, for use in the treatment or prevention of cancer.

In one embodiment, is provided the medical composition as described herein, for use the treatment or prevention of postpartum hemorrhage, menorrhagia, trauma-associated hemorrhage, and surgical bleeding.

In one embodiment, is provided the medical composition as described herein, for use the treatment or prevention of coagulopathy, e.g. traumatic coagulopathy.

In one embodiment, is provided the medical composition as described herein, for use the treatment or prevention of post-inflammatory hyperpigmentation, dermal melanosis, rosacea, and telangiectasia.

The medical composition as described herein is suitably in the form of a topical gel, a topical membrane or a topical patch.

In a further aspect of the invention is provided a kit comprising:
a centrifugation container; and
a container comprising an antifibrinolytic substance.

Embodiments and preferences set out above in relation to the centrifugation container and antifibrinolytic substance hereinabove apply equally to the kit.

In one embodiment of the kit, the centrifugation container comprises:
a density gradient medium; and/or
an anticoagulant; and/or
a coagulation activator; and/or
a structural biomaterial.

The density gradient medium is as described hereinabove, and in particular is a thixotropic gel. The anticoagulant is as described hereinabove, and in particular is sodium citrate. The coagulation activator is as described hereinabove, in particular is calcium gluconate. The structural biomaterial is as described hereinabove, and in particular is heparosan or hyaluronic acid, and is preferably hyaluronic acid. The centrifugation container of the kit optionally comprises a further therapeutic agent selected from the group consisting of a corticosteroid, an NSAID (in particular ketorolac), kartogenin, a haemoglobin, an anaesthetic (as described herein above, in particular bupivacaine), and an analgesic (in particular acetaminophen), or any combination thereof. The antifibrinolytic substance (in the separate container i.e. not present in the centrifugation container) is as described hereinabove, and in particular is tranexamic acid. Suitably the separate container comprising the antifibrinolytic substance (e.g. tranexamic acid) is a syringe. The centrifugation container of the kit is used to prepare platelet rich plasma, to which the antifibrinolytic substance (e.g. tranexamic acid) in the separate container (e.g. syringe) is then added.

It should be noted that in the context of the present application, when referring to a range of between about “AA” and about “BB”, the point values of AA and BB are intended to be included as possible values in the range.

The word “comprise”, and variations such as “comprises” and “comprising” as used herein should be understood to mean the inclusion of the stated integer, step, group of integers or group of steps, but not to the exclusion of any other integer, step, groups of integers or group of steps.

The word “consisting of” as used herein limits the scope of the integer, step, group of integers or group of steps to the specified integer, step, groups of integers or group of steps. The word “consisting essentially of” as used herein limits the scope of the integer, step, group of integers or group of steps, and further integers, steps, groups of integers or groups of steps that do not materially affect the basic and novel characteristics of the invention.

The invention embraces all combinations of indicated integers, steps, groups of integers or groups of steps recited above. All patents and patent applications referred to herein are incorporated by reference in their entirety.

### ABBREVIATIONS

ACD acid citrate dextrose
ACL anterior cruciate ligament
CBD cannabidiol
EDTA ethylenediaminetetraacetic acid
EGF epidermal growth factor
HA hyaluronic acid
HMW-HA high molecular weight hyaluronic acid
LMW-HA low molecular weight hyaluronic acid
LP-PRP leukocyte-poor platelet rich plasma
MCL medial collateral ligament
MPA modified polyamide
MR/LR-PRP monocyte- and lymphocyte-rich platelet rich plasma
MSC mesenchymal stem cells
NSAID non-steroidal anti-inflammatory drug
PBS phosphate buffered saline
PDGF platelet-derived growth factor
PEG polyethylene glycol
PET polyethyleneterephthalate
PRP platelet rich plasma
PRF platelet rich fibrin
TGF transforming growth factor
THC tetrahydrocannabinol
TXA tranexamic acid
VEGF vascular endothelial growth factor

### EXAMPLES

### General Methods

### PDGF Assay

An enzyme-linked immunosorbent assay (ELISA) is used to measure platelet-derived growth factor delivery following platelet activation.

### Example 1—In Vitro Study: Speed of PRP Fibrin Clot Disintegration

Liquid platelet rich plasma in cylindrical moulds (1 cm diameter, 2 mm height) was mixed with tranexamic acid (10 mg or 20 mg), and then mixed with calcium chloride (0.1 mL) to form a PRP gel disk. A control disk without tranexamic acid was also prepared. Fifteen PRP gel discs were divided into three groups of five:
1. Group “TXA-”: pure PRP gel used as a negative control.
2. Group “TXA 10” group: PRP gel with added tranexamic acid (TXA) at a dose of 10 mg/ml.
3. Group “TXA 20” group: PRP gel with added tranexamic acid (TXA) at a dose of 20 mg/ml.

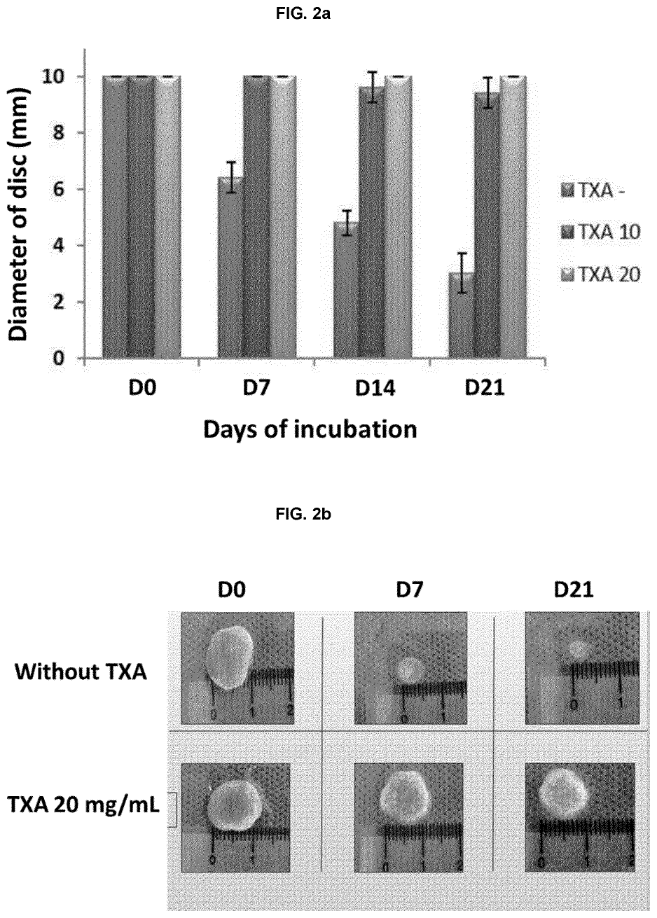

The discs were incubated under physiological conditions and their diameter measured at weekly intervals over a 21 day period. The results are shown in FIGS. 2a and 2b, where D0=0 days; D7=7 days, D14=14 days and D21=21 days. It can be seen that in the presence of tranexamic acid, the rate of PRP gel disc breakdown was significantly reduced. This is indicative of the fibrin matrix within the PRP gel disc being preserved.

### Example 2—In Vitro Study: Measurement of Platelet Growth Factor Release Kinetics

Liquid platelet rich plasma in cylindrical moulds (1 cm diameter, 2 mm height) was mixed with tranexamic acid or adenosine diphosphate (ADP; a platelet aggregation activator), and then mixed with calcium chloride (0.1 mL) to form a PRP gel disk. A control disk without tranexamic acid or ADP was also prepared. Fifteen PRP gel discs were divided into three groups of five:
1. Group “CaCl2”: pure PRP gel used as a negative control.
2. Group “TXA+CaCl2”: PRP gel with added tranexamic acid (TXA).
3. Group “CACl2+ADP”: PRP gel with added ADP.

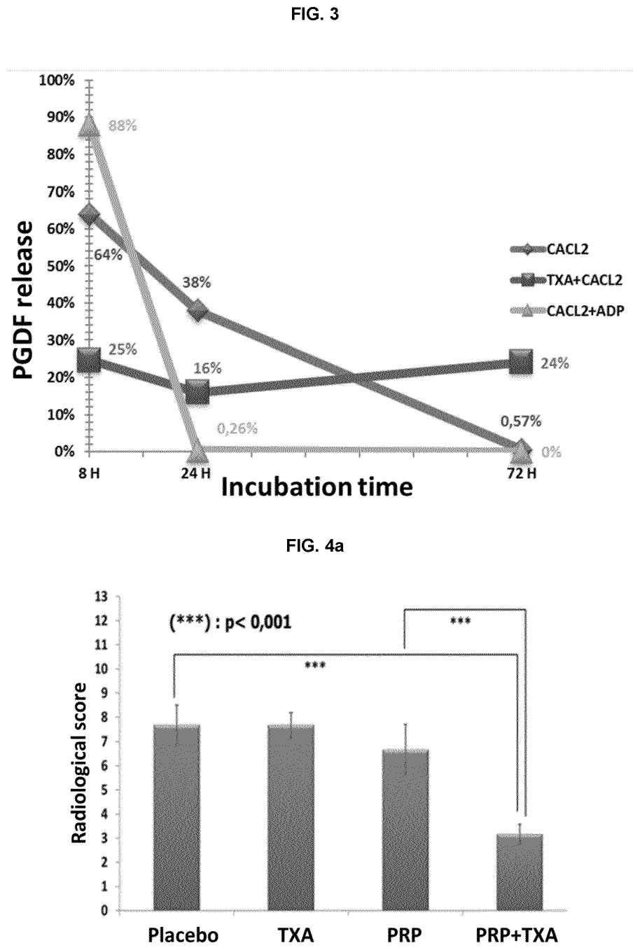

The discs were incubated under physiological conditions for 8, 24 or 72 hours. Following incubation, the discs were subjected to a PDGF assay (see General Methods). The results are shown in FIG. 3, where it can be seen that the preparation containing tranexamic acid exhibited prolonged release of growth factors, compared with the preparations not containing tranexamic acid. It is believed that the slower delivery of growth factors over a longer time period provides optimal biological activity for wound and tissue healing.

### Example 3—In Vivo Study: Arthrosis Rabbit Model

Rabbits showing characteristic signs of osteoarthritis (as described in Guingamp et al., 1997; confirmed with the sacrifice of two rabbits on day 15 after induction of osteoarthritis and histology), were injected with a treatment solution as described below. The rabbits were sacrificed after 75 days and radiological observation, macroscopic observation, histological observation of the cartilage, histological observation of the synovium, histological observation of the menisci were carried out.

The rabbits were treated with either:
1. PRP alone; or
2. TXA (tranexamic acid) alone
3. PRP+TXA (tranexamic acid); or
4. Physiological serum (saline solution, a placebo)

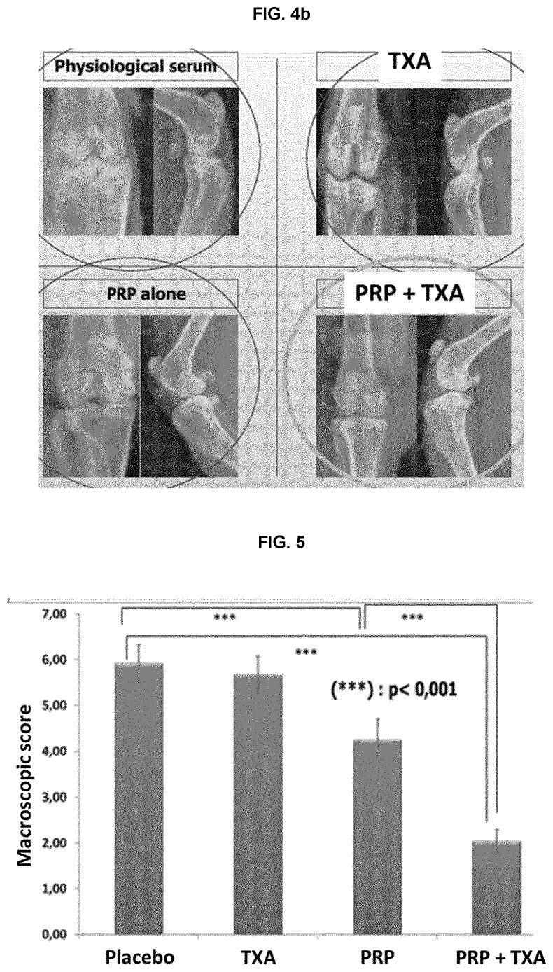

The limbs were examined radiologically by two orthogonal views (anteroposterior and medio-lateral) performed using a SAMSUNG XGO® model GU60 digital radiography system (Samsung® Electronics Co, Ltd; Korè) according to radiological constants following: Voltage: 52 kV; Intensity: 100 mA; and Load: 5 mAs. The radiographs were examined blindly by two observers. The results are shown in FIGS. 4a and 4b. The degenerative changes are similar between the two subgroups of group control (physiological serum and TXA alone). Treatment with PRP alone reduced the intensity of the radiological score compared with the physiological serum, but without reaching the threshold of statistical significance (p=0.092). Treatment with PRP+TXA provided significantly the best radiological score compared with treatment with PRP alone (p=0.0001). This is explained by the fact that tranexamic acid prevents the destruction of the fibrin network within the joint space and allows a prolonged biological action.

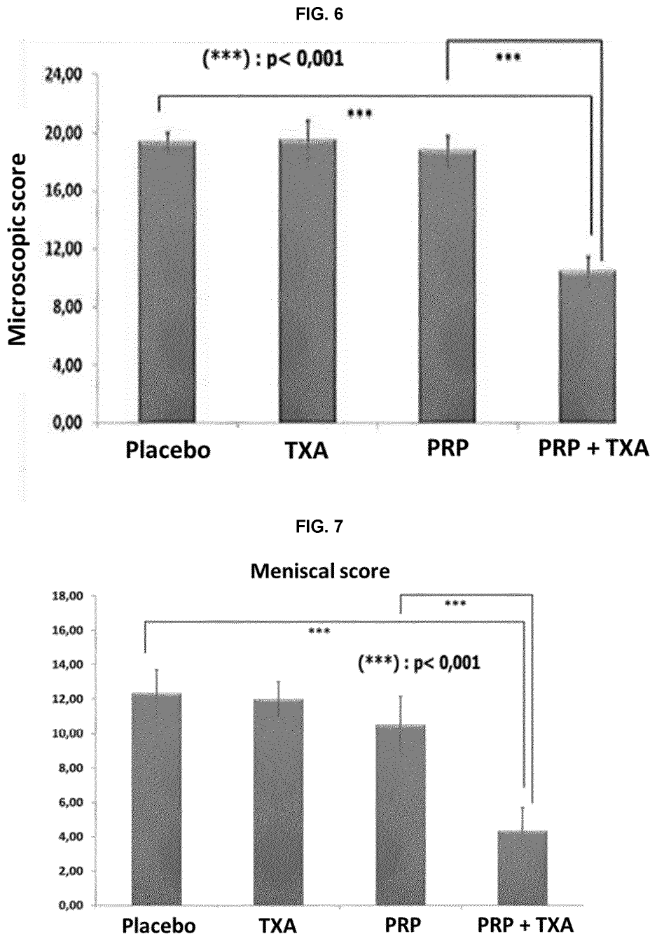

Macroscopic scores are shown in FIG. 5 and microscopic scores (histological evaluation of cartilage) are shown in FIG. 6, wherein it can again be seen that the combination of PRP and tranexamic acid provided the most effective treatment.

Meniscal scores are shown in FIG. 7. The analysis of the data of the histological score of the different meniscus reveals the following results: no statistically significant differences between the “placebo” group and the “TXA” group (p=0.6). Treatment with “PRP alone” has no effect on the degenerative menisci changes compared to “Placebo” (p=0.07). Treatment with “PRP+TXA” significantly improved the damaged meniscus compared to “placebo” (p=2.2*10-6) and “PRP alone” (p=5.6*10-6).

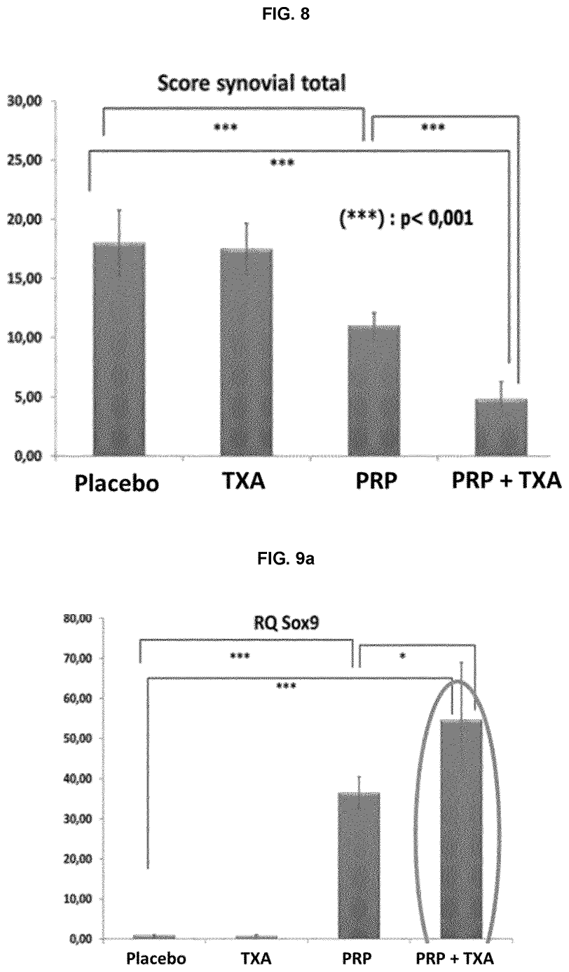

Synovial scores are shown in FIG. 8. For osteoarthritic synoviopathy, the histology data reveal that: no statistically significant differences between the “placebo” group and the “TXA” group (p=0.58). PRP alone reduces osteoarthritic synoviopathy (p=1.7*10-5). Treatment with “PRP+TXA” significantly improved the remodelling of synovial tissue compared to “placebo” (p=1.19*10-6) and “PRP alone” (p=4.9*10-5). These differences are also noted for the different score histological parameters, except for the “synovial inflammation” parameter, where there was no significant difference between the “PRP alone” and “placebo” group.

### Example 4—In Vivo Study: Arthrosis Rabbit Model—Molecular Biology

The measurement of the expression of the genes responsible for the cell differentiation is found to be important in assessing the potential regenerative of the therapy tested. The process of tissue repair involves the overexpression of the genes that are involved in the form of messenger RNA (mRNA) which will be essential for synthesis of proteins necessary for wound healing and differentiation. Therefore, quantification of the mRNA of such a gene will reflect the efficiency and the intensity of tissue repair. Based on this principle, in the rabbit osteoarthritis model of Example 3, RT-PCR was carried out in order to quantify two genes that are correlated to repair and differentiation hyaline cartilage: the COL II gene coding for the synthesis of collagen II; and the SOX9 gene coding for the differentiation mechanisms chondrocyte.

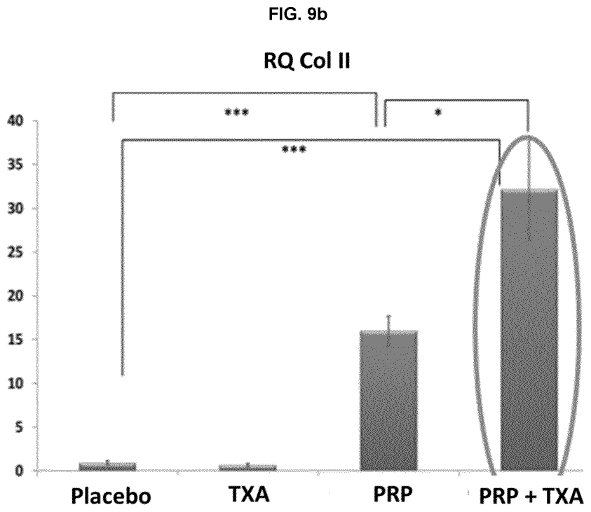

RQ Sox 9 results are shown in FIG. 9a and RQ Col II results are shown in FIG. 9b. No significant difference in the expression of the two SOX9 genes (p=0.36) and COLII (p=0.12) between the “Placebo” group and the “TXA” group. Treatment with “PRP alone” significantly improves the expression of SOX9 gene (p=3.6*10-8) and COLII (p=3.9*10-8) compared to Placebo group. Treatment with “PRP+TXA” significantly improved the expression of SOX9 compared to “placebo” (p=1.3*10-5) and “PRP alone” (p=0.04). Treatment with “PRP+TXA” significantly improved the expression of COLII compared to “placebo” (p=2.1*10-6) and “PRP alone” (p=0.004).

### Example 5—Effect of Structural Biomaterial and Coagulation Activator on PRP Fibrin Clot Disintegration

The following PRP gels were prepared:

### Gel 1. PRP+Autologous Thrombin+Calcium Gluconate;

Platelet rich plasma (PRP) was prepared in a centrifugation tube containing thixotropic gel. Autologous thrombin serum (ATS) was prepared in a centrifugation tube without additional components. A mixture of PRP-ATS was formed, and calcium gluconate (CaGlu) was added (10% of the PRP volume, e.g. 0.3 mL of CaGlu for 3 mL of PRP). The resulting PRP gel was added to a Petri dish.

### Gel 2. PRP+Autologous Thrombin+Calcium Gluconate+Tranexamic Acid;

Platelet rich plasma was prepared in a centrifugation tube containing thixotropic gel. Autologous thrombin was prepared using a centrifugation tube without additional components. A mixture of PRP-ATS was formed, and calcium gluconate (CaGlu) was added (10% of the PRP volume, e.g. 0.3 mL of CaGlu for 3 mL of PRP). Tranexamic acid was added when the clot (PRP-ATS-CaGlu) was formed, in a petri dish, superficially (0.3 mL).

### 3. PRP+Hyaluronic Acid+Calcium Gluconate (Coagulation Activator)

Platelet rich plasma was prepared in a centrifugation tube containing thixotropic gel, hyaluronic acid and calcium gluconate. The resulting PRP gel was added to a Petri dish.

### 4. PRP+Hyaluronic Acid+Calcium Gluconate+Tranexamic Acid

Platelet rich plasma was prepared in a centrifugation tube according to the invention containing a thixotropic gel, sodium citrate, hyaluronic acid, calcium gluconate and tranexamic acid. In the starting tube (prior to blood collection and centrifugation), the sodium citrate was located above the layer of thixotropic gel, which was itself located above a mixture of hyaluronic acid, calcium gluconate and tranexamic acid. The resulting PRP gel was added to a Petri dish.

Plasmin (the enzyme at least in part responsible for degradation of the matrix created into the joint in pathological conditions such as inflammation and knee osteoarthritis, and post-surgery) was applied superficially to each gel. After 3 weeks, it was observed that the texture of the fibrin clot was significantly more stable in gel 4 (PRP+hyaluronic acid+calcium gluconate+tranexamic acid) than in gels 1-3. In particular, the clot in gel 4 was more stable than gel 2, where the tranexamic acid was added only after the PRP had been formed. This is in contrast to gel 4, where the tranexamic acid was present in the centrifugation tube from the outset and could exert early stage action.

### Example 6—Preparation of a Composition for Intra-Articular Injection

A 10 mL (125 mm) centrifugation tube with the following components was prepared:

| Thixotropic gel | 3 | g |
|---|---|---|
| Sodium citrate dihydrate | 24 mg (4% = 40 mg/mL) | |
| Tranexamic acid | 90 mg (15% = 150 mg/mL) | |
| Water for injection | 0.6 | mL |

The separating gel is located at the bottom of the tube, with the solution of sodium citrate dihydrate and tranexamic acid on top of the layer of thixotropic gel.

Whole blood (approximately 5 mL) is drawn into the tube, which is then centrifuged at a force of between about 1500 g and about 2000 g. The resulting platelet rich plasma solution contains tranexamic acid at a concentration of about 16 mg/mL. This solution has potential utility as an intra-articular injection for the treatment of, for example, osteoarthritis e.g. of the knee.

### Example 7—Preparation of a Composition for Topical Administration

A 10 mL (125 mm) centrifugation tube with the following components was prepared:

| Thixotropic gel | 3 | g |
|---|---|---|
| Sodium citrate dihydrate | 24 mg (2.4% = 24 mg/mL) | |
| Tranexamic acid | 125 mg (12.5% = 125 mg/mL) | |
| Water for injection | 1 | mL |

The separating gel is located at the bottom of the tube, with the solution of sodium citrate dihydrate and tranexamic acid on top of the layer of thixotropic gel.

Whole blood (approximately 5 mL) is drawn into the tube, which is then centrifuged at a force of between about 1500 g and about 2000 g. The resulting platelet rich plasma solution contains tranexamic acid at a concentration of about 21 mg/mL. This solution has potential utility as a topical composition for the treatment of, for example, a wound.

### REFERENCES

Assirelli et al. Knee Surg. Sports Traumatol. Arthrosc. 2015, 23, 2690-2703.
Brancato et al. Am. J. Pathol. 2011, 178, 19-25.
Chen et al., J. Biophotonics. 2012, 5, 777-784.
Guingamp et al. Arthritis Rheum. 1997; 40 (9): 16709.
Iio et al. Asia-Pacific Journal of Sports Medicine, Arthroscopy, Rehabilitation and Technology 2016, 4, 27-32.
Knighton et al. Ann. Surg. 1982 196, 379-388.
Kuckelman et al. J. Trauma Acute Care Surg. 2018 July; 85 (1): 91-100
Mann et al. Arterioscler. Thromb. Vasc. Biol. 2003, 23, 17-25.
Marx et al. J. Oral Maxillofac. Surg. 2004, 62, 489-496
Lo et al. JAMA 2003, 290, 3115-3121.
Schaffer et al. Br. J. Surg. 1998, 85, 444-460.
Smith et al. Microvasc. Res. 2007, 73, 84-94.
Woo et al. J. Am. Acad. Orthop. Surg. 2000 November-December; 8 (6): 364-72.

What is claimed is:

1. A centrifugation container comprising an antifibrinolytic substance.

2. (canceled)

3. The centrifugation container according to claim 1, further comprising a density gradient medium is a thixotropic gel.

4-16. (canceled)

17. The centrifugation container according to claim 1, wherein the antifibrinolytic substance is tranexamic acid, aminocaproic acid or mixture thereof.

18. The centrifugation container according to claim 17, wherein the antifibrinolytic substance is tranexamic acid.

19. The centrifugation container according to claim 18, wherein the antifibrinolytic substance is 5 wt. % tranexamic acid.

20-21. (canceled)

22. The centrifugation container according to claim 3, wherein the antifibrinolytic substance is located below the density gradient medium, or at a more distal end of the container than the density gradient medium.

23. (canceled)

24. The centrifugation container according to claim 1, further comprising an anticoagulant, which is suitably selected from the group consisting of sodium citrate, acid citrate dextrose (ACD), modified ACD, heparin or a salt thereof, ethylenediaminetetraacetic acid (EDTA) or a salt thereof, an iodo acetate salt, an oxalate salt, and a fluoride salt.

26-29. (canceled)

30. The centrifugation container according to claim 24, wherein the anticoagulant is located above the density gradient medium, or at a more proximal end of the container than the density gradient medium; and the antifibrinolytic substance is located below the density gradient medium, or at a more distal end of the container than the density gradient medium.

31-42. (canceled)

43. The centrifugation container according to claim 1, further comprising a structural biomaterial, wherein the structural biomaterial is selected from the group consisting of a glycosaminoglycan, a silk protein, fibroin, collagen, polysaccharide and a polylactide, or any combination thereof.

44. The centrifugation container according to claim 43, wherein the structural biomaterial is a glycosaminoglycan, such as selected from the group consisting of chondroitin, chondroitin sulfate, dermatan, dermatan sulfate, heparin, heparan sulfate, heparosan, hyaluronan and hyaluronic acid.

45. (canceled)

46. The centrifugation container according to claim 44, wherein the glycosaminoglycan is heparosan or hyaluronic acid, and in particular is hyaluronic acid.

47-53. (canceled)

54. The centrifugation container according to claim 43, wherein the structural biomaterial is located below the density gradient medium, or at a more distal end of the container than the density gradient medium.

55. The centrifugation container according to claim 43, wherein the ratio of structural biomaterial to antifibrinolytic substance (v/v) is between about 10:1 and about 500:1, such as between about 5:1 and about 250:1; and in particular is about 15:1.

56. (canceled)

57. The centrifugation container according to claim 1, further comprising one or more agents selected from the group consisting of a steroid, a corticosteroid, a glucocorticosteroid, a non-steroidal anti-inflammatory drug (NSAID), kartogenin, an anaesthetic, and antibacterial compound, an antibiotic, an antifungal compound, an antiparasitic compound, an enzyme, an enzyme inhibitor, a glycoprotein, a growth factor, a hormone, an antiviral compound, an analgesic, an opioid, a saponin, a haemoglobin, tetrahydrocannabinol (THC), cannabidiol (CBD), an anti-angiogenetic agent, anti-melanogenetic agent, an immunomodulator, an immunoglobulin, a mineral, a neuroleptic, a protein, a peptide, a lipoprotein, a tumouricidal compound, a tumourstatic compound, a toxin, a vitamin (such as vitamin A, vitamin E, vitamin B, vitamin C, vitamin D; or a derivative thereof) or a wrinkle filler, or a combination thereof, in particular a further therapeutic agent is selected from the group consisting of a corticosteroid, an NSAID, kartogenin, a haemoglobin, an anaesthetic, an analgesic, an opioid, a saponin and THC, or a combination thereof.

58-59. (canceled)

60. The centrifugation container according to claim 1, comprising, consisting essentially of or consisting of an antifibrinolytic substance, a density gradient medium, an anticoagulant and hyaluronic acid.

61. (canceled)

62. The centrifugation container according to claim 60, wherein the antifibrinolytic substance is tranexamic acid, the density gradient medium is a thixotropic gel, and the anticoagulant is sodium citrate.

63-65. (canceled)

66. A process for preparing a medical composition, comprising the steps of:
(a) centrifuging whole blood or bone marrow in a centrifugation container according to claim 1; and then
(b) collecting the medical composition.

67. A medical composition obtained according to the process of claim 66.

68-104. (canceled)

105. A medical composition comprising platelet rich plasma (PRP) and an antifibrinolytic substance, prepared using a centrifugation container according to claim 1.

106. The medical composition according to claim 105, wherein the antifibrinolytic substance is tranexamic acid.
